(19) United States
(12) Patent Application Publication
(10) Pub. No.: US 2013/0035553 A1
(43) Pub. Date: Feb. 7, 2013

(54) Endoscope with multiple working channel ports

(71) Applicant: Gregory S. Konstorum, Stamford, CT (US)

(72) Inventor: Gregory S. Konstorum, Stamford, CT (US)

(73) Assignee: Gyrus ACMI, Inc.

(21) Appl. No.: 13/136,463

(22) Filed: Aug. 1, 2011

(51) Int. Cl. A61B 1/12; B23P 17/04

(52) U.S. Cl. 600/156; 29/428

(57) ABSTRACT

An endoscope including a shaft having a distal end with a lens; a control section at a rear end of the shaft; and a working channel. The control section includes a frame having two working channel ports on opposite respective lateral sides of the control section, where each port forms a single aperture into the control section provided by an outwardly extending fitting. The fittings are configured to have an irrigation conduit connected thereto. The working channel ports are angled from rear to front in a forward and inward direction. The working channel has a rear end connected to both of the working channel ports such that an instrument can be positioned into the working channel through either of the two working channel ports. The working channel extends from the control section through the shaft as a single working channel for both of the working channel ports.

### BACKGROUND

1. Technical Field

The exemplary and non-limiting embodiments of the invention relate generally to an endoscope and, more particularly, to a working channel in an endoscope.

2. Brief Description of Prior Developments

Japanese Patent Publication No. 08-238212 discloses an endoscope with two working channels each having a respective forceps opening. U.S. Patent Publication no. 2007/0118016 A1 discloses an endoscope with a conventional port and an additional port in the shaft.

### SUMMARY

The following summary is merely intended to be exemplary. The summary is not intended to limit the scope of the claims.

In accordance with one aspect, an endoscope is provided comprising a shaft, a control section at a rear end of the shaft, and a working channel. The shaft comprises at least a portion of the the working channel, and illumination and image members. The control section comprises a frame having two working channel ports on opposite respective lateral sides of the control section. Each port forms a single aperture into the control section provided by an outwardly extending fitting. Each of the fittings are configured to have an irrigation conduit connected thereto. The two working channel ports are both angled from rear to front in a forward and inward direction along a length of the control section. The working channel has a rear end connected to both of the working channel ports in the control section such that an instrument can be positioned into the working channel through either of the two working channel ports. The working channel extends from the control section through the shaft as a single working channel for both of the working channel ports.

In accordance with another aspect, an endoscope is provided comprising a shaft comprising a distal end having a lens; a control section at a rear end of the shaft; and a working channel. The control section comprises a frame having two working channel ports and a channel junction connected to the two working channel ports. The working channel extends from the channel junction through the shaft. A rear end of the working channel is connected to the channel junction. Front ends of the two working channel ports are connected to the channel junction. The two working channel ports have a substantially same size from an exterior of the control section to the channel junction. The two working channel ports are each configured to alternatively have an instrument inserted through the port, through the channel junction and into the working channel.

In accordance with another aspect, a method comprises connecting a rear end of a first working channel of an endoscope to a channel junction; and connecting front ends of two working channel ports to the channel junction, where the two working channel ports have a substantially same size, and where the channel junction is sized and shaped to provide access to the first working channel from both of the working channel ports through the channel junction such that an instrument can be positioned into the working channel through either of the two working channel ports.

### BRIEF DESCRIPTION OF THE DRAWINGS

The foregoing aspects and other features are explained in the following description, taken in connection with the accompanying drawings, wherein:

FIG. 1 is a perspective view of an example embodiment;

FIG. 2 is a side view of the apparatus shown in FIG. 1;

FIG. 3 is a perspective view of the control section of the apparatus shown in FIGS. 1-2 from another direction;

FIG. 4 is a front view of the control section shown in FIGS. 1-3

FIG. 5 is a sectional view of some of the components of the control section shown in FIG. 4 taken along line 5-5;

FIG. 6 is a perspective view of the junction member shown in FIG. 5;

FIG. 7 is a rear side view of the junction member shown in FIG. 6;

FIG. 8 is a bottom side view of the junction member shown in FIG. 6;

FIG. 9 is a right side view of the junction member shown in FIG. 6;

FIG. 10 is a top side view of the junction member shown in FIG. 6;

FIG. 11 is a front side view of the junction member shown in FIG. 6;

FIG. 12 is a cross sectional view of the junction member shown in FIG. 11 taken along line 12-12; and

FIG. 13 is a cross sectional view of the junction member shown in FIG. 11 taken along line 13-13.

### DETAILED DESCRIPTION OF EMBODIMENT

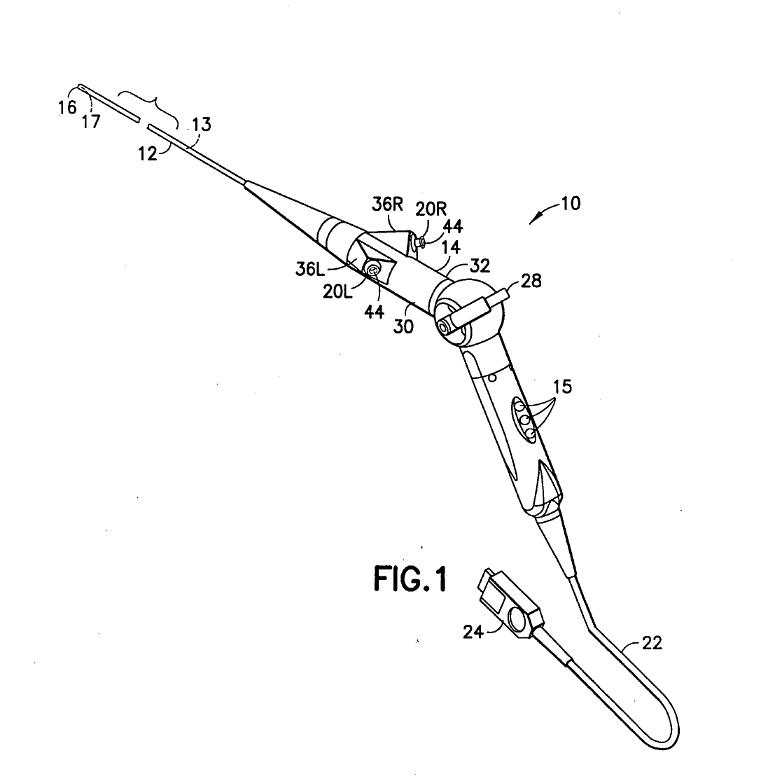

Referring to FIG. 1, there is shown a perspective view of an endoscope 10 incorporating features of an example embodiment. Although the features will be described with reference to the example embodiment shown in the drawings, it should be understood that features can be embodied in many alternate forms of embodiments. In addition, any suitable size, shape or type of elements or materials could be used.

The endoscope 10, in this example embodiment, is a medical instrument used for inspection inside a patient. The endoscope is an optical instrument connected to a video unit, and comprises a working channel for delivery and removal of a distention or irrigation medium, and providing access of a tool through the endoscope. However, features of the invention could be used in any suitable type of endoscope.

In the example shown, the endoscope comprises a shaft 12 and a main section or control section 14 which forms a handle. The shaft 12 has a front or distal end 16 which is controllably deflectable. The rest of the shaft could be substantially rigid, or might be flexible or semi-flexible. The distal end 16 is adapted to deflection in up and down directions. However, any suitable deflection capability could be provided, including left and right directions for example.

The control section 14 is located at the proximal end of the shaft 12. The control section 14 has two working channel ports or inlets; 20L on the left side and 20R on the right side. The ports 20L, 20R are configured to allow an instrument and fluid, such as irrigation fluid for example, to pass into the working channel. The control section 14 also has a cable 22 with a connector 24 adapted to be connected to another device. An electrical cable extends to a video sensor at the distal end 16 and light emitting diodes (LEDs). Alternatively, fiber optic bundles for illuminating and imaging in front of the distal end 16 could be provided. In either case the distal end 16 comprises a lens 17.

Deflection control wires or cables (not shown) extend to the distal end 16 from a pulley with control lever 28 in the control section 14. The pulley with control lever 28 is rotatably mounted in the frame 30 of the control section 14. The pulley with control lever 28 and deflection control wires form part of a deflection control for a user to control deflection of the deflectable distal end 16 from the control section 14. As seen in FIG. 1, in this embodiment the control section 14 has buttons 15 to control features of another device which the connector 24 is connected to.

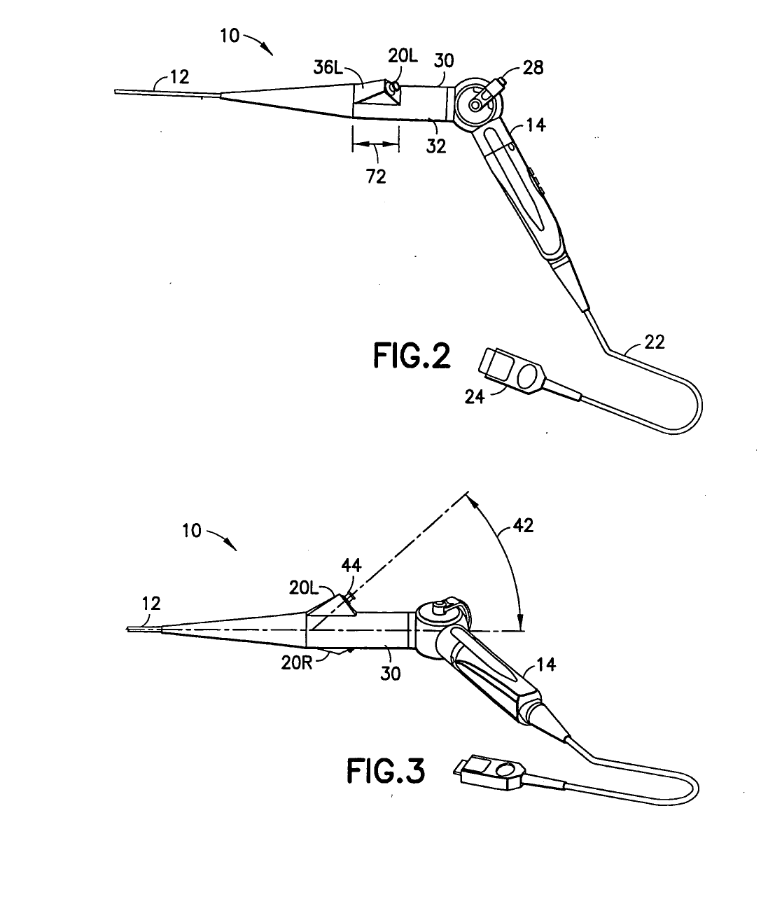

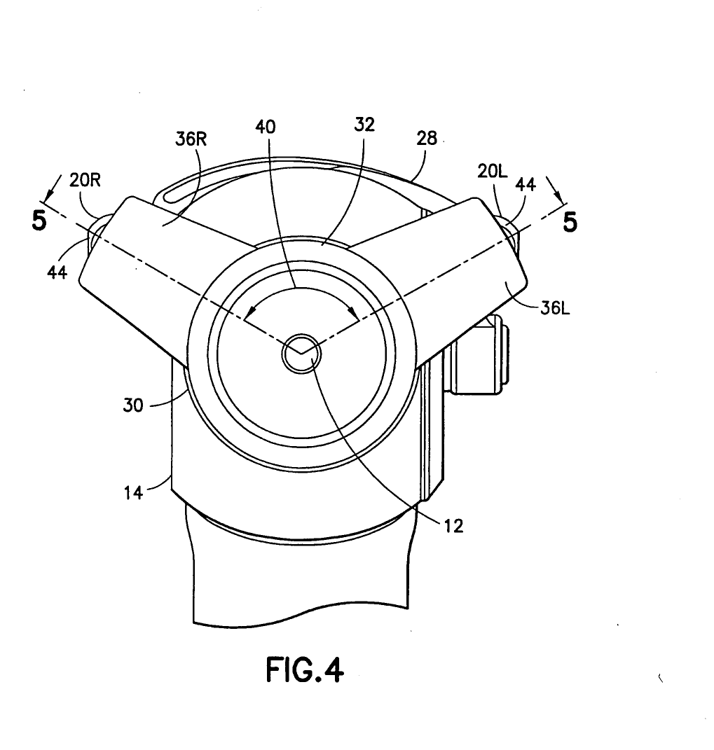

Referring also to FIGS. 2-5 the frame 30, among other members, comprises a one-piece metal member 32. The member 32 includes a center channel 34 (see FIG. 5) and two outwardly extending mound sections 36L, 36R on the exterior of the frame. Each mound section has a hole 38 therethrough from a rear of the mound section into the center channel 34. One mound section 36L is located on a left side of the control section 14. The other mound section 36R is located on the right side of the control section 14. The two mound sections 36L, 36R are angled relative to each other at an angle 40 (see FIG. 4) of about 120 degrees. However, in alternate embodiments the angle 40 might be between 90-150 degrees for example. The mound sections 36L, 36R are located on opposite respective lateral sides of the control section 14. The mound sections 36L, 36R are substantial mirror images of each other at a same longitudinal length position 72 on the frame 30. The holes 38 are both angled relative to a centerline of the member 32 by an angle 42 (see. FIG. 3) of about 40 degrees. However, in alternate embodiments the angle 42 might be between 20-60 degrees.

Referring particularly to FIG. 5, the ports 20L, 20R are formed by the mound sections 36L, 36R in addition to fittings 44 at each mound section. The fittings 44 are identical. However, in alternate embodiments the fittings could be different. Each fitting 44 is mounted in a respective one of the holes 38. Each fitting 44 includes a front end 46, a rear end 48, a front seal 50 and a rear seal 52. A conduit 54 is provided through the fitting between the rear end 48 and the front end 46. The rear end 48 extends out from the hole 38 and is sized and shaped to allow an irrigation conduit or tube (not shown) to be attached to the rear end 48. The rear seal 52 makes a sealing engagement between the frame of the fitting and the member 32 in the hole 38.

The front ends 46 of the two fittings 44 are connected to a single working channel 26 by a channel junction 56. The channel junction 56 is a one-piece member. However, in an alternate embodiment more than one member could be combined to form the channel junction. A funnel member 60 connects the rear end of the working channel 26 to the front end of the channel junction 56.

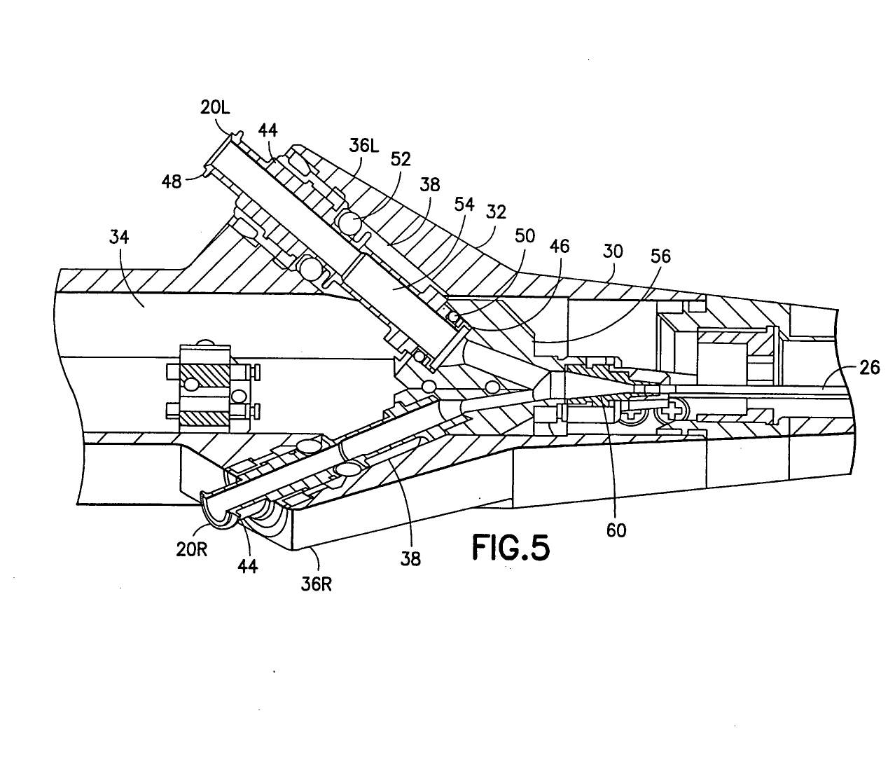

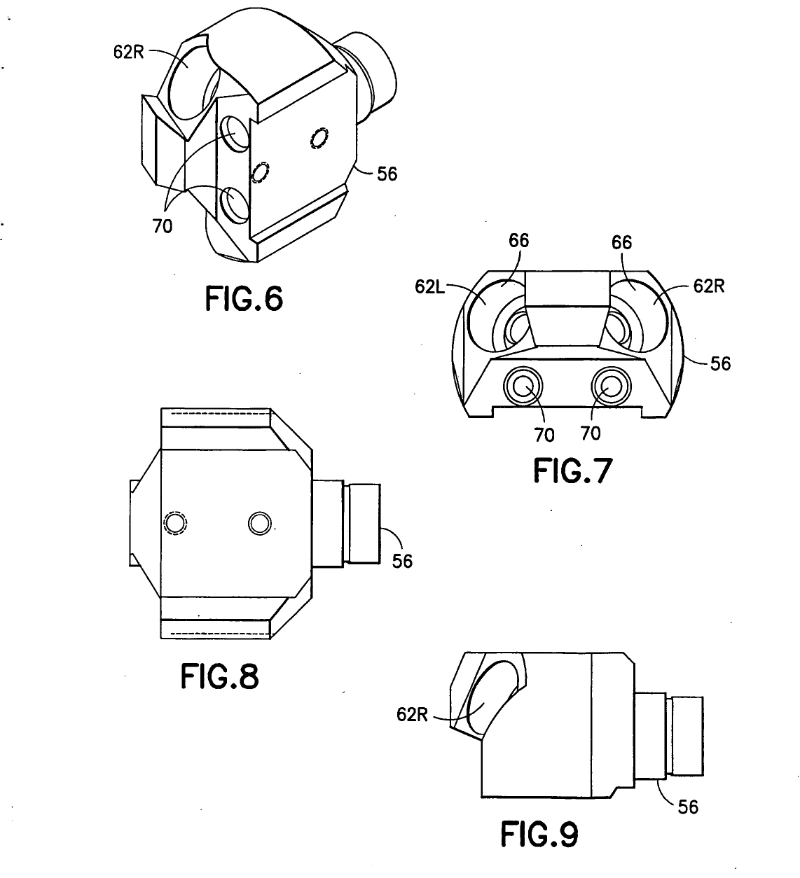

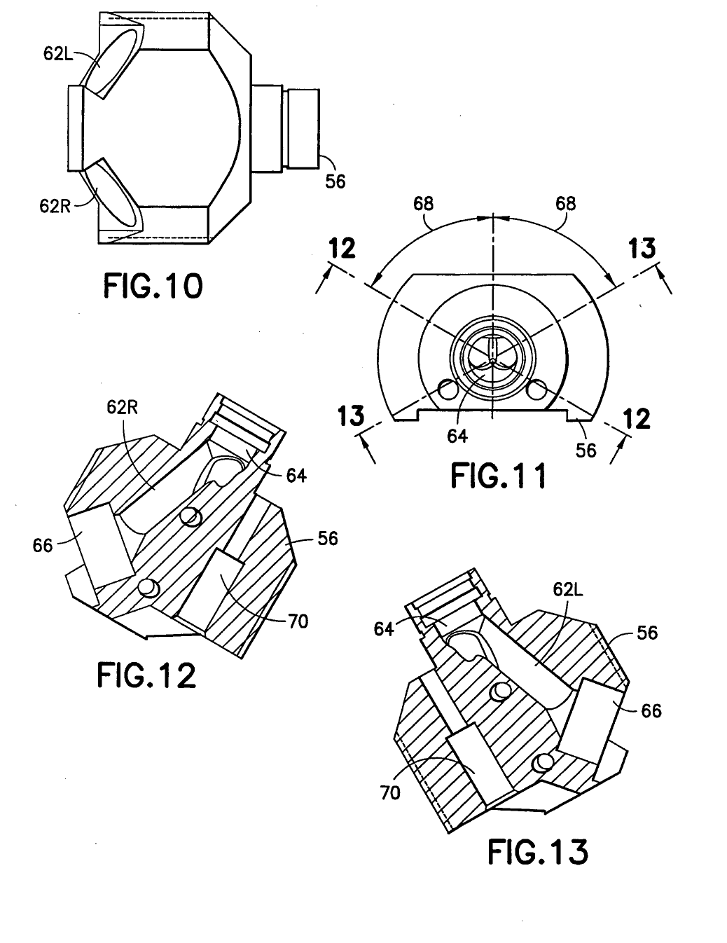

FIGS. 6-13 show various views of the one-piece channel junction 56. The channel junction 56 comprises two rear channels 62L, 62R and a single front channel 64. The front channel 64 receives the funnel member 60 and extends in a general straight direction parallel to the centerline of the frame member 32. The two rear channels 62L, 62R have front ends which merge with the rear end of the front channel 64. The two rear channels 62L, 62R are angled relative to the front channel 64, from rear to front, downward, forward and laterally inward similar to the holes 38 in the frame member 32. In this embodiment angles 68 are about 60 degrees each. Rear ends 66 of the rear channels 62L, 62R are larger than the rest of the rear channels, are angled relative to the rest of the rear channels, and are sized and shaped to have the front ends 46 of respective ones of the fittings 44 mounted therein. Front seals 50 can seal the engagement of the front ends 46 inside the rear ends 66. In this example the junction 56 comprises channels 70 to allow for passage of other members, such as deflection control wires, electrical wires or fiber optics for example.

With the example embodiment described above, the control section 14 can be provided with two substantially identical working channel ports 20L, 20R have a same size and are at a same longitudinal length position 72 on the control section, but at different lateral sides of the control section; one 20R on the right and one 20L on the left. Each port 20L, 20R can receive either an instrument/tool (to be inserted into the single working channel 26) or an irrigation conduit. Thus, the endoscope 10 can be used by either a left-handed user or a right-handed user the same way. A left-handed user might insert the instrument/tool in the left port 20L and attach the irrigation conduit to the rear end 48 of the fitting 44 at the right port 20R. A right-handed user might insert the instrument/tool in the right port 20R and attach the irrigation conduit to the rear end 48 of the fitting 44 at the left port 20L.

Conventional endoscopes with one working channel have a port connected to the working channel. During a surgical procedure the working channel is used for insertion of different tools or accessories, such as a guide wire, scissors, graspers, etc. The working channel is also used for irrigation in order to clean a field of view for example. Because the working channel might be used for both location of an accessory and for irrigation at the same time, the working channel port may have an additional irrigation port or irrigation conduit mount located on the external side of the accessory port such as shown in U.S. Pat. No. 6,749,560 and U.S. Patent Publication No. 2010/0312055 A1 for example. However, this type of construction may jeopardize cleaning and disinfection if done improperly. There is also a disposable Y-type connector which may be attached to a port not having its own irrigation conduit mount. However, use of a disposable Y-type connector requires longer accessories and increase waste. Some flexible endoscopes, such as described in U.S. Pat. No. 4,750,477 for example, have a working channel port attached to a rotatable block to improve the scope ergonomics for left-handed and right-handed users. This makes it easier to use for a left or right hand surgeon by merely rotating the block (and its working channel port) to the left or to the right, but this rotatable block solution complicates design and assembly.

The example described above can provide two working channel ports connected to a single working channel, where each port is located on respective left and right sides of the housing. Both ports are exactly the same, and each one can be used for accessories insertion by a left or right hand surgeon; with the remaining second port being available for connection of an irrigation conduit. With this type of construction, unlike a conventional endoscope having a working channel port with an additional irrigation port or irrigation conduit mount located on the external side of the accessory port, cleaning and disinfection is not jeopardized even if done improperly. With this type of construction, unlike a conventional endoscope using a disposable Y-type connector, it does not require longer accessories and does not increase waste. With this type of construction, unlike a conventional endoscope using rotatable block to improve the scope ergonomics for left-handed and right-handed users, it does not complicate design and assembly as much as a rotatable block.

An example embodiment can provide an endoscope 10 comprising a shaft 12 comprising fiber optic or video member(s) 13 and a distal end 16 having a lens 17; a control section 14 at a rear end of the shaft; and a working channel 26. The control section 14 comprises a frame 30 having two working channel ports 20L, 20R on opposite respective lateral sides of the control section. Each port 20 forms a single aperture into the control section provided by an outwardly extending fitting 44. Each of the fittings 44 are configured to have an irrigation conduit connected thereto. The two working channel ports 20 are both angled from rear to front in a forward and inward direction along a same length 72 (see FIG. 2). of the control section. The working channel 26 has a rear end connected to both of the working channel ports 20 in the control section 14 such that an instrument can be positioned into the working channel through either of the two working channel ports. The working channel 26 extends from the control section 14 through the shaft 12 as a single working channel for both of the working channel ports 20.

The control section 14 can comprise a channel junction 56 mounted inside the frame 30, where the channel junction comprises two rear channels 62 extending into a single front channel 64. The channel junction 56 may be a one-piece member. Front ends 46 of the fittings 44 may be mounted in respective ones of the rear channels 62. The rear end of the working channel 26 may be connected to the front channel 64. A funnel member 60 may be located in the front channel and connect the rear end of the working channel to the front channel. The two working channel ports 20L, 20R may be substantially mirror images of each other on the opposite respective lateral sides of the control section. Each of the two working channel ports 20 may be angled from rear to front relative to a centerline of the control section at an angle of about 40 degrees. The two working channel ports 20 may be angled about 120 degrees apart relative to each other. The two working channel ports 20 may have rear ends 48 with the single aperture which are located at a same longitudinal length position on the frame.

An example embodiment can provide an endoscope comprising a shaft comprising fiber optic or video members with a distal end having a lens; a control section at a rear end of the shaft and a working channel. The control section can comprise a frame having two working channel ports and a channel junction connected to the two working channel ports. The working channel can extend from the channel junction through the shaft, where a rear end of the working channel is connected to the channel junction. Front ends of the two working channel ports can be connected to the channel junction, where the two working channel ports have a substantially same length from an exterior of the control section to the channel junction. The two working channel ports may be configured to alternatively have an instrument inserted through the port, through the channel junction and into the working channel.

The channel junction may comprise a one-piece member mounted inside the frame, where the channel junction comprises two rear channels extending into a single front channel, where front ends of the working channel ports are connected to respective ones of the two rear channels. A funnel member may be located in the front channel of the channel junction and connects the rear end of the working channel to the front channel. The working channel ports may each comprise the frame having an outwardly extending mound section on an exterior of the frame; a hole extending through the mound section into an interior of the frame; and a fitting mounted in a respective one of the holes, where the fitting forms an area adapted to attached an irrigation conduit.

The two working channel ports may both be angled from rear to front in a forward and inward direction along a same length of the control section. The two working channel ports may be substantially mirror images of each other on opposite respective lateral sides of the control section. The two working channel ports may each be angled from rear to front relative to a centerline of the control section at an angle of about 20-60 degrees, where the two working channel ports are angled about 90-150 degrees apart relative to each other, and where the two working channel ports have rear ends with an aperture into each port which are located at a same longitudinal length position on the frame.

Another example may provide a method comprising connecting a rear end of a first working channel of an endoscope to a channel junction; and connecting front ends of two working channel ports to the channel junction, where the two working Channel ports have a substantially same length, and where the channel junction is sized and shaped to provide access to the first working channel from both of the working channel ports through the channel junction such that an instrument can be positioned into the working channel through either of the two working channel ports. The working channel ports may comprise holes through a frame of a control section of the endoscope, and a fitting of each of the working channel ports is inserted into each one of the holes to form the ports, where front ends of the fittings are directly connected to the channel junction, and where rear ends of the fittings extend out from the frame. The method may comprise providing a frame of a control section of the endoscope with outwardly extending mound sections on opposite lateral exterior sides of the frame at a same longitudinal length position on the frame, providing a hole extending through each of the mound sections into an interior of the frame, and mounting a respective fitting in each of the holes, where the fittings each form an area adapted to attached an irrigation conduit, such that the instrument can be inserted into either of the two working channel ports and an irrigation conduit can be attached to the other one of the ports.

It should be understood that the foregoing description is only illustrative. Various alternatives and modifications can be devised by those skilled in the art. For example, features recited in the various dependent claims could be combined with each other in any suitable combination(s). In addition, features from different embodiments described above could be selectively combined into a new embodiment. Accordingly, the description is intended to embrace all such alternatives, modifications and variances which fall within the scope of the appended claims.

What is claimed is:

1. An endoscope comprising:
a shaft comprising a distal end having a lens;
a control section at a rear end of the shaft, where the control section comprises a frame having two working channel ports on opposite respective lateral sides of the control section, where each port forms a single aperture into the control section provided by an outwardly extending fitting, where each of the fittings are configured to have an irrigation conduit connected thereto, where the two working channel ports are both angled from rear to front in a forward and inward direction along a length of the control section; and
a working channel having a rear end connected to both of the working channel ports in the control section such that an instrument can be positioned into the working channel through either of the two working channel ports, where the working channel extends from the control section through the shaft as a single working channel for both of the working channel ports.

2. An endoscope as in claim 1 where the control section comprises a channel junction mounted inside the frame, where the channel junction comprises two rear channels extending into a single front channel.

3. An endoscope as in claim 2 where the channel junction is a one-piece member.

4. An endoscope as in claim 2 where front ends of the fittings are mounted in respective ones of the rear channels.

5. An endoscope as in claim 4 where the rear end of the working channel is connected to the front channel.

6. An endoscope as in claim 5 where a funnel member is located in the front channel and connects the rear end of the working channel to the front channel.

7. An endoscope as in claim 1 where the two working channel ports are substantially mirror images of each other on the opposite respective lateral sides of the control section.

8. An endoscope as in claim 7 where the two working channel ports are each angled from rear to front relative to a centerline of the control section at an angle of about 40 degrees.

9. An endoscope as in claim 8 where the two working channel ports are angled about 120 degrees apart relative to each other.

10. An endoscope as in claim 1 where the two working channel ports have rear ends with the single aperture which are located at a same longitudinal length position on the frame.

11. An endoscope comprising:
a shaft comprising a distal end having a lens;
a control section at a rear end of the shaft, where the control section comprises a frame having two working channel ports and a channel junction connected to the two working channel ports; and
a working channel extending from the channel junction through the shaft, where a rear end of the working channel is connected to the channel junction, where front ends of the two working channel ports are connected to the channel junction, where the two working channel ports have a substantially same length from an exterior of the control section to the channel junction, and where the two working channel ports are each configured to alternatively have an instrument inserted through the port, through the channel junction and into the working channel.

12. An endoscope as in claim 11 where the channel junction comprises a one-piece member mounted inside the frame, where the channel junction comprises two rear channels extending into a single front channel, where front ends of the working channel ports are connected to respective ones of the two rear channels.

13. An endoscope as in claim 12 where a funnel member is located in the front channel of the channel junction and connects the rear end of the working channel to the front channel.

14. An endoscope as in claim 11 where the working channel ports each comprise:
the frame having an outwardly extending mound section on an exterior of the frame;
a hole extending through the mound section into an interior of the frame; and
a fitting mounted in a respective one of the holes, where the fitting forms an area adapted to attached an irrigation conduit.

15. An endoscope as in claim 11 where the two working channel ports are both angled from rear to front in a forward and inward direction along a same length of the control section.

16. An endoscope as in claim 11 where the two working channel ports are substantially mirror images of each other on opposite respective lateral sides of the control section.

17. An endoscope as in claim 11 where the two working channel ports are each angled from rear to front relative to a centerline of the control section at an angle of about 20-60 degrees, where the two working channel ports are angled about 90-150 degrees apart relative to each other, and where the two working channel ports have rear ends with an aperture into each port which are located at a same longitudinal length position on the frame.

18. A method comprising:
connecting a rear end of a first working channel of an endoscope to a channel junction; and
connecting front ends of two working channel ports to the channel junction, where the two working channel ports have a substantially same length, and where the channel junction is sized and shaped to provide access to the first working channel from both of the working channel ports through the channel junction such that an instrument can be positioned into the working channel through either of the two working channel ports.

19. A method as in claim 18 where the working channel ports comprise holes through a frame of a control section of the endoscope, and a fitting of each of the working channel ports is inserted into each one of the holes to form the ports, where front ends of the fittings are directly connected to the channel junction, and where rear ends of the fittings extend out from the frame.

20. A method as in claim 18 further comprising providing a frame of a control section of the endoscope with outwardly extending mound sections on opposite lateral exterior sides of the frame at a same longitudinal length position on the frame, providing a hole extending through each of the mound sections into an interior of the frame, and mounting a respective fitting in each of the holes, where the fittings each form an area adapted to attached an irrigation conduit, such that the instrument can be inserted into either of the two working channel ports and an irrigation conduit can be attached to the other one of the ports.
